(19) United States
(12) Patent Application Publication
(10) Pub. No.: US 2026/0306866 A1
(43) Pub. Date: Oct. 8, 2026

(54) COMPACT WEARABLE PULSED ELECTROMAGNETIC FIELD THERAPY SYSTEM FOR BONE HEALING WITH IMPLANT FIELD CONCENTRATION

(71) Applicant: Wurth Medical Innovations, LLC, Brentwood, TN (US)

(72) Inventors: Todd R. Wurth, Brentwood, TN (US); Barry R. Hix, Birmingham, AL (US); Thomas J. Bachinski, Lakeville, MN (US)

(73) Assignee: Wurth Medical Innovations, LLC, Brentwood, TN (US)

(21) Appl. No.: 19/566,734

(22) Filed: Mar. 13, 2026

Related U.S. Application Data
(63) Continuation-in-part of application No. 19/202,837, filed on May 8, 2025.
(60) Provisional application No. 63/644,868, filed on May 9, 2024.

(51) Int. Cl. A61N 2/00; A61N 2/02; A61N 2/06

(52) U.S. Cl. CPC A61N 2/004; A61N 2/002; A61N 2/02; A61N 2/06

(57) ABSTRACT

Disclosed herein is a compact wearable pulsed electromagnetic field (PEMF) therapy system. The system may be used for bone healing and optimized for activation of implantable passive electromagnetic components. The system may comprise an external PEMF generator having a compact electromagnetic coil, pulse generation circuit, and wearable housing. In one application, the generator operates in implant-activation mode (about 3-10 mT) to activate OsteoCore implanted passive components—permanent magnets, with or without integrated electromagnetic induction coils, integrated into standard orthopaedic hardware—positioned directly at a bone fracture site. Advantageous, the system may eliminate geometric field divergence inherent in conventional circumferential external PEMF systems, enabling 40-60% coil diameter reduction and 60-85% weight reduction while achieving targeted field delivery at the fracture site. The compact design may enable truly wearable form factors improving patient compliance. The system may also be architecturally configured for a standalone PEMF generation mode (about 8-20 mT) independent of implanted components, which may have application as a clinical bone growth stimulator. In one embodiment, the system may incorporate magnetic field-based bone healing monitoring, wherein changes in external coil impedance resulting from tissue magnetic susceptibility changes during healing enable non-invasive assessment of healing progress without additional implanted sensors or batteries. Further embodiments may employ conformable coil architectures including curved rigid coils, flexible continuous coils on flex-PCB substrates, and segmented articulated coil arrays enabling conformance to varying limb anatomies. Some embodiment may include selective coil element activation using magnetometer-based proximity detection optimizes field delivery efficiency. The system may be connectable to smartphones, which may enable compliance tracking and remote patient management.

### CROSS-REFERENCE TO RELATED APPLICATIONS

This application claims priority to U.S. Provisional Application No. 63/644,868, filed May 9, 2024, U.S. Non-Provisional application Ser. No. 19/202,837, filed May 8, 2025, titled “Medical Hardware Energy Delivery System.” The entire disclosures of U.S. Provisional Application No. 63/644,868 and U.S. Non-Provisional application Ser. No. 19/202,837 are hereby incorporated herein by reference.

### FIELD OF INVENTION

The present invention relates generally to electromagnetic therapy systems for bone healing, and more particularly, to compact, wearable pulsed electromagnetic field (PEMF) generators, which may be optimized for use with implantable passive electromagnetic components, and to dual-mode PEMF systems, which may be capable of operating both as implant activators and as standalone PEMF generators.

### BACKGROUND

Bone fractures represent a significant healthcare burden, with approximately 6-8 million fractures occurring annually in the United States alone. While most fractures heal with standard immobilization or surgical fixation, 5-30% of cases experience delayed healing or nonunion, particularly in high-risk patient populations including the elderly, diabetics, smokers, and patients with osteoporosis or compromised vascular supply.

Electromagnetic field therapy has been established as an effective adjunctive treatment for bone healing for over 45 years. Two primary modalities exist:
(a) Pulsed Electromagnetic Field (PEMF) Therapy: External devices generate time-varying magnetic fields that induce electric currents in bone tissue via electromagnetic induction (Faraday's law). PEMF devices have been FDA-cleared since 1979 (e.g., Physio-Stim K881658, OrthoGen K944782) for treatment of fresh fractures, delayed unions, and nonunions.
(b) Capacitively Coupled Electric Field (CCEF) Therapy: Devices generate electric fields in tissue through capacitive coupling. CCEF therapy has been FDA-cleared since 1984 for spinal fusion and nonunion treatment (e.g., EBI Bone Healing System K033522).

Both PEMF and CCEF therapies have demonstrated efficacy in multiple randomized controlled trials, with clinical evidence showing 10-30% improvement in union rates and 2-4 week reductions in healing time compared to controls.

### Conventional External PEMF Devices—Design Characteristics

Current FDA-cleared external PEMF bone growth stimulators (Physio-Stim, OrthoGen, Orthopak) employ a circumferential coil architecture in which the treatment coil must be centered around the fracture site. The device instructions for use language for each cleared device explicitly states that the coils must be centered around the fracture site to be effective, and Orthofix markets the Physio-Stim's 360-degree field coverage as a device feature. This circumferential architecture requires:
Large electromagnetic coils (15-25 cm diameter) to encircle the treatment anatomy
High power consumption (20-50 watts) to achieve adequate field strength at depth when field energy is distributed circumferentially
Heavy, bulky form factors (2-4 kg total weight)
Wearable form factors that remain large and bulky due to the circumferential coil architecture requirement, limiting patient acceptance and comfort during extended daily wear
Size and weight (2-4 kg total) that create practical barriers to consistent daily wear compliance

These design constraints result in poor patient compliance. Published literature reports compliance rates of only 60-80% with conventional external PEMF devices, with non-compliance primarily attributed to device size, weight, discomfort during extended wear, and the circumferential positioning requirement which limits practical use in daily activities.

### Conventional Implantable CCEF Devices—Limitations

Active implantable CCEF devices (EBI Bone Healing System, SpinalPak) address compliance issues but introduce different limitations, such as:
Battery implantation requiring surgical dissection
Battery removal surgery at 6-9 months post-implantation
Infection risk at battery pocket site (1-3% in published literature)
Skin erosion over battery packs in thin patients
Patient anxiety regarding implanted electronics

Thus, opportunities exist for further improvements and advancements in such devices to enhance functionality and patient compliance.

### BRIEF SUMMARY

Incorporated herein are energy delivery systems that are operable to generate and deliver magnetic and/or electric fields to a target area proximate to an implantation site. Such systems may include implantable passive electromagnetic components comprising stacked neodymium magnets, with or without integrated electromagnetic induction coils, housed within a biocompatible encapsulation and integrated into standard orthopaedic hardware.

The implantable passive electromagnetic components may operate through, at least, two mechanisms when exposed to external PEMF fields:
(1) Positioning electromagnetic therapy delivery directly at the fracture site, eliminating geometric field divergence inherent in external-only PEMF systems where the therapeutic coil is positioned 5-10 cm from the fracture site and
(2) Electromagnetic induction generating capacitively coupled electric fields (CCEF) via Faraday induction, delivering combined PEMF+CCEF therapy without requiring implanted batteries or active electronics.

Disclosed herein is an extension of this system by providing external PEMF generator designs, which may be optimized to harness the full therapeutic potential of implanted components positioned directly at fracture sites.

External PEMF generators may be the energy source for activating the implanted passive components. The external PEMF generators may be specifically optimized for use with implanted passive components, which may enable compact wearable form factors. Conventional PEMF devices require large circumferential coil geometries and high power to achieve therapeutic field strength at fracture sites 5-10 cm from the external coil. When passive electromagnetic components are positioned directly at fracture sites, these geometric constraints may be replaced by optimized compact designs delivering targeted field energy to the implant site.

Disclosed herein are external PEMF generator designs specifically optimized for use with implanted passive electromagnetic components. These designs take advantage of reduced geometric field divergence requirements to enable compact, truly wearable form factors not achievable with conventional external-only circumferential PEMF systems.

Also disclosed herein are dual-mode PEMF systems. These systems may be capable of operating as activators for patients with implanted passive electromagnetic components (“OsteoCore components”) and/or as standalone PEMF generators for independent operation. Standalone operation as a bone growth stimulator for patients without implanted components represents a future clinical application pathway subject to independent clinical validation and regulatory clearance, as described herein.

### BRIEF DESCRIPTION OF EMBODIMENTS

Disclosed herein are compact wearable pulsed electromagnetic field (PEMF) therapy systems. Said systems may be specifically optimized for use with implantable passive electromagnetic components. Also disclosed herein are dual-mode systems architecturally capable of both implant activation and/or standalone PEMF generation.

In a first aspect, provided herein is a wearable PEMF therapy system. The system may comprise an external PEMF generator including an electromagnetic coil (may have a diameter of about 8-18 cm), pulse generation circuit (may have frequencies of about 1-200 Hz), power source (may have about 2-15 watts), and housing (may have a mass of about 200-1200 grams). Such an embodiment may represent a 40-60% coil diameter reduction, 70-90% power reduction, and 60-85% weight reduction compared to conventional external circumferential PEMF devices, achieved through implant-optimized design eliminating the need for large circumferential coil geometries.

In a second aspect, provided herein is a dual-mode PEMF therapy system. The system may be configured to operate in: (a) an implant-activation mode generating targeted pulsed magnetic fields of about 3-10 mT to activate implanted passive electromagnetic components positioned at a fracture site; and (b) a standalone PEMF generation mode producing pulsed magnetic fields of about 8-20 mT for operation independent of implanted components.

In a third aspect, provided herein is a bone healing monitoring system. The system may utilize implanted permanent magnets as passive sensors. Changes in the impedance of an external measurement coil resulting from tissue magnetic susceptibility changes during fracture healing may enable non-invasive assessment of healing progress without additional implanted sensors, batteries, or active electronics.

In a fourth aspect, provided herein is conformable coil architectures. The architectures may include curved rigid coils, flexible continuous coils on flex-PCB substrates, and segmented articulated coil arrays comprising multiple rigid coil elements connected by flexible electrical conductors, enabling the coil array to conform to anatomies of varying circumference.

### BRIEF DESCRIPTION OF THE DRAWINGS

So that the manner where the above recited features may be understood in detail, a more particular description, briefly summarized above, may be had by reference to example aspects, some of which are illustrated in the appended drawings.

FIG. 1 is a side-by-side comparison diagram illustrating the architectural distinction between a conventional circumferential external PEMF device (left panel) and an embodiment of a compact unilateral OsteoCore-paired PEMF generator of the present invention (right panel), showing relative coil diameter, device weight, and coil placement geometry.

FIG. 2 is a schematic diagram illustrating dual-mode operation of an embodiment of a compact PEMF generator: Panel A shows implant-activation mode with OsteoCore implanted components at the fracture site; Panel B shows standalone PEMF generation mode without implanted components.

FIG. 3 is a perspective view of an embodiment of a compact disc-shaped wearable PEMF generator showing a housing, electromagnetic coil (dashed), elastic strap attachment, user interface buttons, and USB-C charging port.

FIG. 4 is a cross-sectional view of the compact disc-shaped PEMF generator of FIG. 3, showing internal component arrangement including an electromagnetic coil, battery, electronics PCB, housing, and strap attachment points.

FIG. 5 is a schematic diagram of an embodiment of a smartphone-connected PEMF therapy system showing wireless communication between the wearable PEMF generator, paired smartphone application, and cloud-based healthcare provider portal.

FIG. 6 is a cross-sectional diagram of a patient limb illustrating electromagnetic field delivery from an embodiment of a compact external PEMF generator through tissue to OsteoCore implanted passive components at the fracture site, showing field convergence and therapeutic field zone concentration at the implant location.

FIG. 7 is a comparative diagram illustrating the U.S. annual fracture patient population addressable by the OsteoCore-paired implant platform versus the broader platform architecture addressable market including future standalone PEMF generation applications pending clinical validation.

FIG. 8 is a block diagram of the electronic architecture of an embodiment of a compact PEMF generator, showing the microcontroller, power amplification stage, current sensing circuit, battery, voltage regulator, charging circuit, electromagnetic coil, and BLE wireless communication module.

FIG. 9 is a flowchart illustrating a method of delivering and monitoring OsteoCore-paired PEMF therapy, including surgical implantation, post-operative device deployment, compliance logging, remote provider review, and healing-based therapy cessation decision.

FIG. 10 is a schematic diagram of an embodiment of a magnetic field-based bone healing monitoring system, illustrating impedance measurement between the external coil and implanted permanent magnets, magnetometer-based positioning confirmation, and healing stage classification timeline.

FIG. 11 is a line graph illustrating relative inductance change over time post-surgery, showing three healing stage zones corresponding to soft callus formation, mineralizing callus, and mature bone formation, with threshold values subject to clinical validation.

FIG. 12 is a comparative diagram illustrating three embodiments of conformable coil architectures: Panel A, curved rigid coil; Panel B, flexible continuous coil on flex-PCB substrate; and Panel C, segmented articulated coil array, each shown in flat and limb-wrapped configurations.

FIG. 13 is a detailed view of an embodiment of a segmented articulated coil array showing the overall flat configuration, cross-section of a single coil element, cross-section of an articulation joint, and the device wrapped around a forearm.

FIG. 14 is a diagram illustrating universal sizing capability of a segmented articulated coil array on four limb sizes, showing circumferential coverage ranging from approximately 340 degrees on a small wrist to approximately 100 degrees on a large thigh, with therapeutic field delivery at the OsteoCore implant site in each configuration.

FIG. 15 is a diagram illustrating three selective coil element activation modes for a segmented articulated coil array: Panel A, all-panel activation; Panel B, proximity-based selective activation using magnetometer detection of the OsteoCore implant; and Panel C, sequential scanning activation.

FIG. 16 is a diagram illustrating integration of an embodiment of a conformable PEMF device into a textile compression garment, showing the garment flat with integrated pocket, device insertion, completed assembly worn on the lower leg, and cross-sectional view showing device positioning relative to tissue layers and fracture site.

### DETAILED DESCRIPTION

Disclosed herein are compact external PEMF generator systems. The systems may be specifically designed to activate OsteoCore implanted components. One clinical application of the present invention is activation of OsteoCore implanted components in patients undergoing operative fracture fixation with OsteoCore-enabled hardware. A secondary architectural capability standalone PEMF generation independent of implanted components-is described herein as a device functionality, which may have application as a clinical bone growth stimulator.

### II. Compact Wearable External PEMF Generator

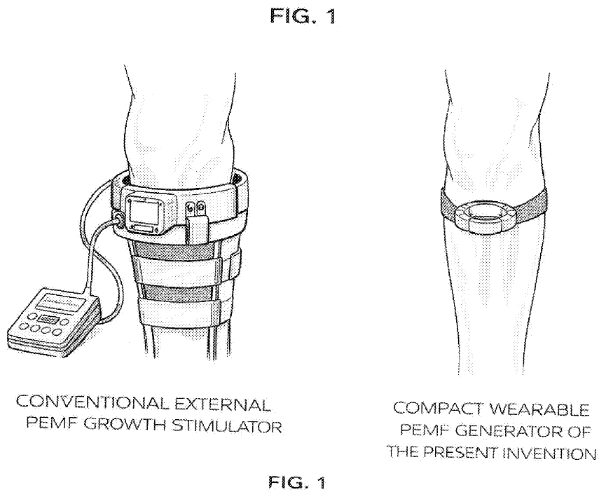

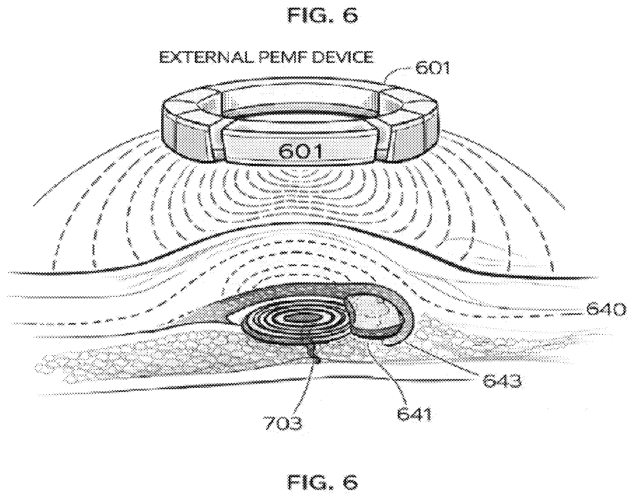

Disclosed herein is a compact wearable external PEMF generator, which may be specifically optimized for activation of OsteoCore implanted passive electromagnetic components. As illustrated in FIG. 1, the architectural distinctions between conventional circumferential PEMF devices and an embodiment of a compact wearable external PEMF generator are shown. Conventional devices require a large coil to encircle the fracture site, while the illustrated embodiment of a compact generator disclosed herein is positioned unilaterally over the OsteoCore implant site. The optimization rationale is as follows: conventional external PEMF devices employ circumferential coil architectures requiring the coil to encircle the fracture site, necessitating large coil diameters (15-25 cm) and high power output (20-50 watts) to achieve adequate field strength at depth. When OsteoCore passive electromagnetic components are positioned directly at the fracture site within the implanted hardware, the external generator need only deliver sufficient field strength at the skin surface to drive the implanted concentrators. The implanted components accomplish the therapeutic field concentration at the fracture site, as shown in FIG. 6. In some embodiments, the compact wearable external PEMF generator comprises the following design parameters:
Coil diameter: about 8 to about 18 cm (40-60% reduction vs. conventional circumferential devices)
Power consumption: about 2 to about 15 watts (70-90% reduction)
Total device mass: about 200 to about 1200 grams (60-85% reduction)
External magnetic field output: about 3 to about 10 mT (implant-activation mode)

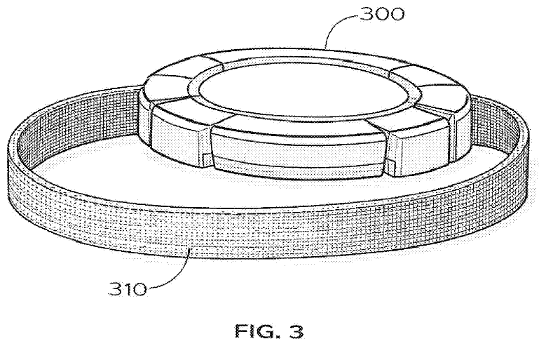

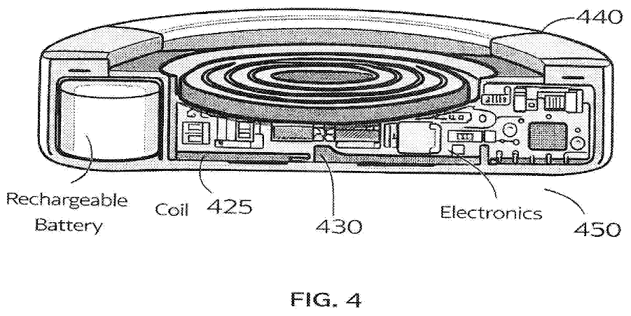

A representative embodiment of the compact wearable PEMF generator is shown in perspective view in FIG. 3. The compact wearable PEMF generator may comprise, for example, one or more of the following: a housing (which may be disc-shaped or any other shape), an electromagnetic coil, an attachment for affixing the housing to a body (which may be an elastic strap attachment), user interface elements, charging port (which may be a USB-C port), and other features. FIG. 4 provides a cross-sectional view of the compact wearable PEMF generator shown in FIG. 3 that shows one embodiment of an internal component arrangement within the housing, which may contain, for example, one or more of the following: electromagnetic coil, a battery pack, and electronics, such as a printed circuit board.

### III. Architecture

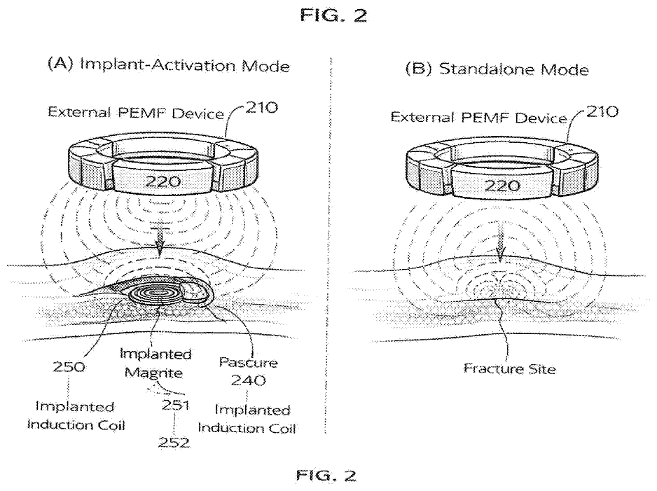

The compact external PEMF generator may be configured to operate in any number or variety of modes. In one embodiment, such as that illustrated schematically in FIG. 2, the compact external PEMF generator may be configured to operate in two modes. The two modes may be an implant-activation mode and a standalone PEMF generation mode. Descriptions of the two exemplary modes are provided below:

Mode A—Implant-Activation Mode: The generator may operate at about 3-10 mT external field output, about 2-8 watts power consumption, and with about a 24-hour battery life per charge cycle. This mode may be designed for patients with OsteoCore implanted components, where the external generator activates the passive concentrators positioned at the fracture site. Clinical performance in this mode is consistent with published large animal model data.

Mode B—Standalone PEMF Generation Mode: The generator may be architecturally capable of operating at about 8-20 mT external field output, about 5-15 watts power consumption, and may not require implanted OsteoCore components. This mode represents a hardware capability of the device. This mode may advantageously stimulate bone growth, even independent of OsteoCore implanted components.

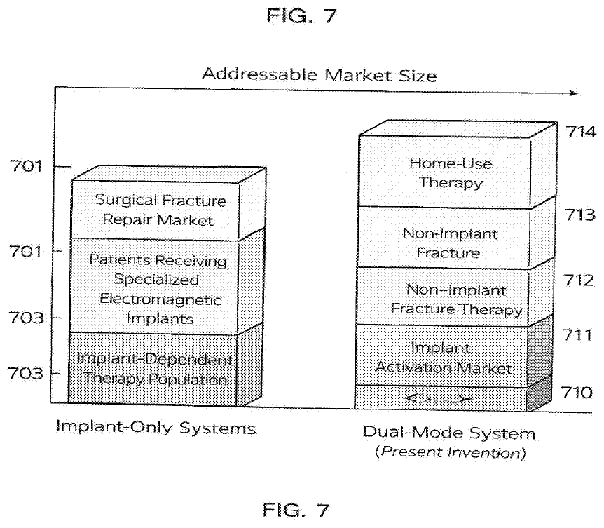

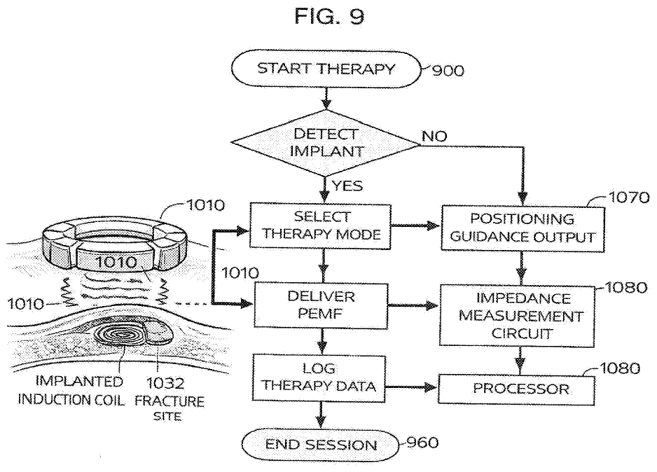

In the dual-mode embodiment, the architecture provides engineering flexibility enabling a single device platform to serve the primary OsteoCore-paired indication while preserving optionality for clinical applications through the standalone generation capability. FIG. 7 illustrates the comparative addressable patient population between the OsteoCore-paired platform and the dual-mode architecture embodiment. FIG. 9 is a flowchart illustrating an embodiment of the OsteoCore-paired therapy delivery and monitoring method, which is also envisioned herein.

### IV. Bone Healing Monitoring System

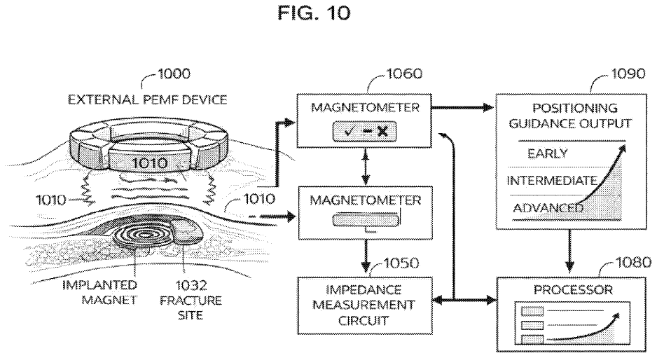

Disclosed herein is a bone healing monitoring system. The bone healing monitoring system may utilize implanted OsteoCore permanent magnets as passive sensors, such as the embodiment illustrated in FIG. 10.

In one embodiment, the monitoring system comprises the following steps:
(a) The external coil applies a low-amplitude interrogation signal at a frequency distinct from the therapeutic PEMF signal.
(b) The impedance of the external coil is measured during the interrogation signal. Coil impedance is influenced by the magnetic coupling between the external coil and the implanted permanent magnets, which in turn is modulated by the magnetic susceptibility of the surrounding tissue.
(c) As fracture healing progresses, tissue magnetic susceptibility changes: early-stage soft callus has different magnetic properties than intermediate mineralizing callus and advanced mature bone. These changes produce measurable changes in external coil impedance over the healing timeline.
(d) Healing stage classification is derived from the measured impedance change relative to baseline: Early healing stage (<0.5% ΔL/L₀), Intermediate healing stage (0.5-1.0% ΔL/L₀), Advanced healing stage (>1.0% ΔL/L₀).

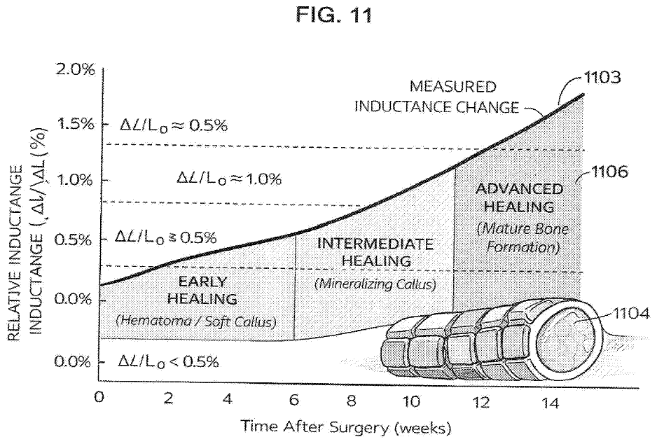

The impedance threshold values recited herein represent initial engineering estimates, as shown graphically in FIG. 11. Specific threshold values for clinical use will be vary and may be refined through clinical validation studies correlating external coil impedance measurements with radiographic healing scores (e.g., RUST scale) in human subjects.

### V. Coil Architectures

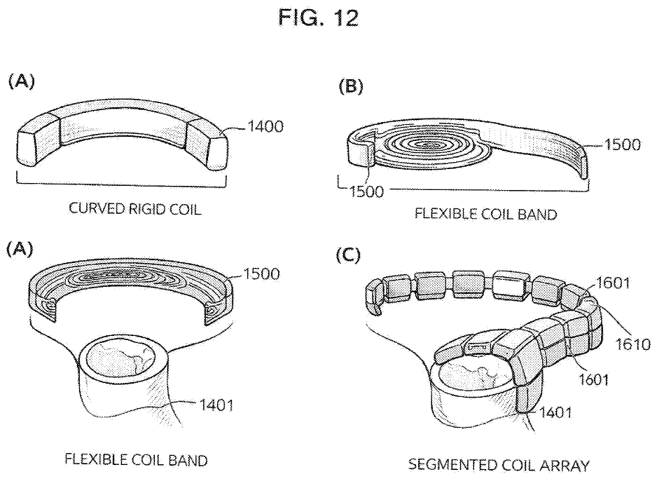

Disclosed herein are coil architectures. The coil architectures may adapt to a patient's body, including but not limited to the curved external anatomy of a patient's limb. The architectures may enable consistent therapeutic positioning across anatomical variations, as illustrated by the comparative embodiments of FIG. 12. Advantageously, such designs may be comfortable for the patient and, thereby, increase patient use and satisfaction.

Three exemplary embodiments are detailed below:

Architecture A—Curved Rigid Coil: A rigid coil housing having a concave patient-contact surface with radius of curvature between about 3 cm and about 15 cm, enabling conformal fit against rounded limb surfaces. This architecture is illustrated in FIG. 12, Panel A.

Architecture B—Flexible Continuous Coil (Flex-PCB): An electromagnetic coil fabricated on a flexible printed circuit board (which may be any substrate, e.g., polyimide or liquid crystal polymer substrate, about 50 to about 200 μm thickness) configured as an elongated band capable of wrapping around limb circumferences from about 15 cm to about 65 cm. This architecture is illustrated in FIG. 12, Panel B.

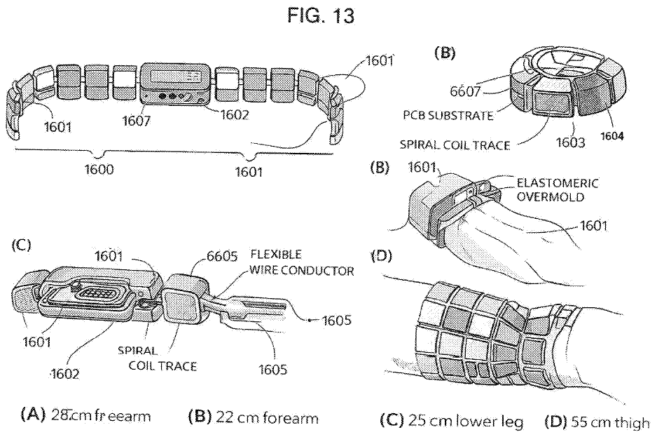

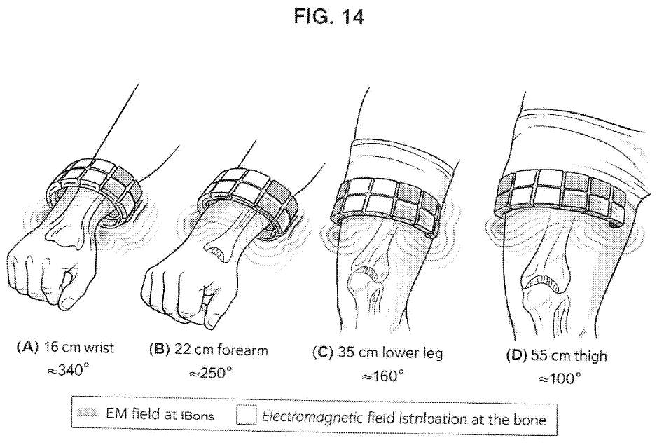

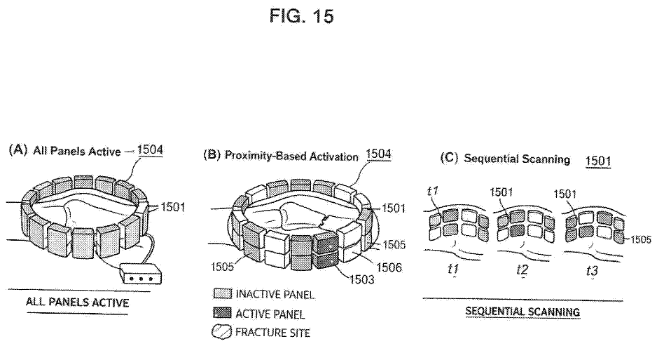

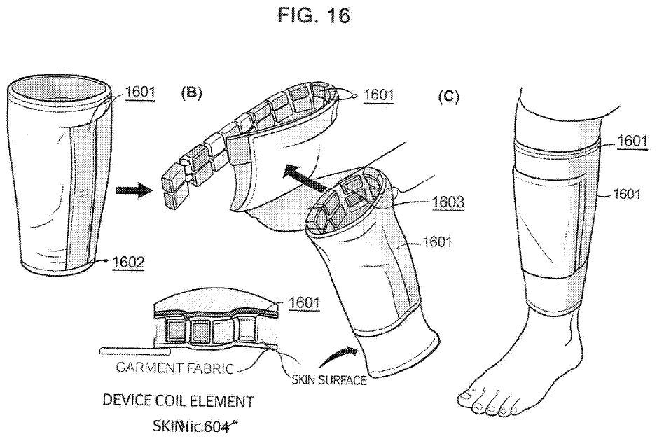

Architecture C—Segmented Articulated Coil Array: A plurality of discrete rigid coil elements (about 6-16 elements, each about 2×3 cm to about 4×6 cm) connected by flexible electrical conductors providing both electrical continuity and mechanical articulation between adjacent elements. Cumulative articulation across multiple joints enables the array to conform to limb circumferences from about 15 cm to about 55 cm. Selective activation of individual coil elements based on magnetometer-detected proximity to implanted components optimizes field delivery efficiency. This architecture is illustrated in FIG. 12, Panel C, with detailed structural views provided in FIG. 13. FIG. 14 illustrates universal sizing capability across four representative limb circumferences. FIG. 15 illustrates three selective coil activation modes. FIG. 16 illustrates integration of the conformable device into a textile compression garment.

### VI. Electronic Architecture and Connectivity

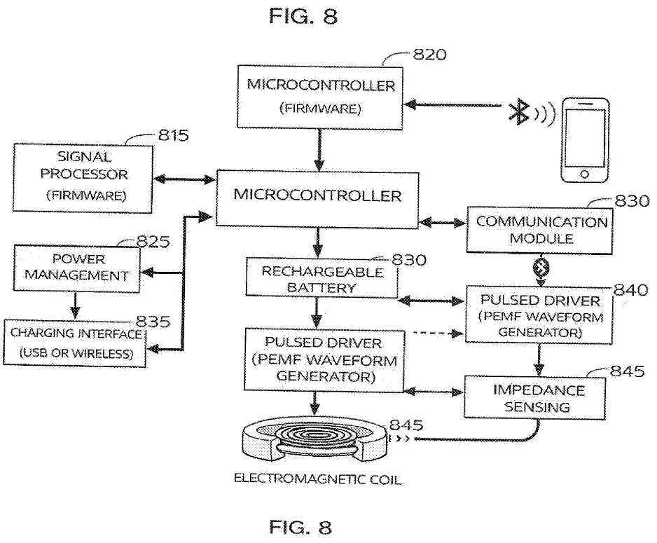

The electronic architecture of the compact PEMF generator may comprise any number of components (e.g., coils, electronics, and so on). In one embodiment and as illustrated in the block diagram of FIG. 8, the electronic architecture comprises: microcontroller unit (MCU) for signal generation and device management; power amplification stage driving the electromagnetic coil; current sensing circuit for real-time output monitoring; a battery (e.g., a rechargeable lithium-ion battery (about 8-20 Wh capacity)); charging circuit (e.g., with a USB-C interface); and a communication module (e.g., a Bluetooth Low Energy (BLE) wireless communication module).

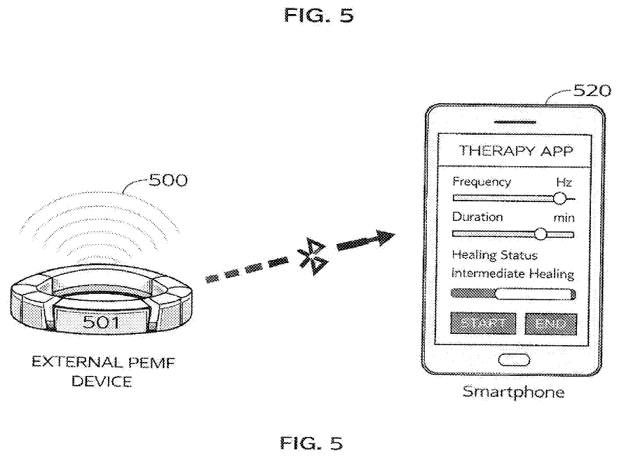

In embodiments having a communication module (such as a BLE module), the module enables bidirectional communication with, for example, a paired smartphone application, as illustrated in FIG. 5. This offers may benefits, such as a compliance tracking with daily therapy hour logging; therapy progress visualization; remote monitoring capability for healthcare providers; and alert notifications for device status and charging requirements.

What is claimed is:

1. A compact wearable pulsed electromagnetic field (PEMF) therapy system for use with implanted passive electromagnetic components, comprising:
(a) an external PEMF generator comprising: (i) an electromagnetic coil having a diameter between 8 cm and 18 cm; (ii) a pulse generation circuit electrically coupled to said electromagnetic coil and configured to generate time-varying magnetic fields at frequencies between 1 Hz and 200 Hz; (iii) a rechargeable power source; (iv) a wearable housing encapsulating said coil, pulse generation circuit, and power source, said housing having a total mass between 200 grams and 1200 grams;
(b) wherein said external PEMF generator is configured to generate an external magnetic field having a flux density between 3 mT and 10 mT for activating implantable passive electromagnetic components positioned at a bone fracture site;
(c) a wearable attachment mechanism configured to position said external PEMF generator on the patient's skin overlying the fracture site during normal daily activities.

2. A dual-mode pulsed electromagnetic field (PEMF) therapy system comprising:
(a) an external PEMF generator configured to operate selectively in: (i) an implant-activation mode generating external magnetic fields between 3 mT and 10 mT at power consumption between 2 watts and 8 watts, for activating implantable passive electromagnetic components positioned at a bone fracture site; and (ii) a standalone PEMF generation mode generating external magnetic fields between 8 mT and 20 mT at power consumption between 5 watts and 15 watts, for operation independent of implanted components;
(b) a mode selection mechanism enabling user selection between said implant-activation mode and said standalone PEMF generation mode;
(c) wherein said external PEMF generator has a total mass between 200 grams and 1200 grams and a maximum coil dimension between 8 cm and 18 cm.

3. (canceled)

4. A bone healing monitoring system comprising:
(a) implanted permanent magnets positioned at a bone fracture site within orthopaedic hardware;
(b) an external measurement coil positioned on the patient's skin overlying said fracture site;
(c) an impedance measurement circuit configured to measure the impedance of said external measurement coil;
(d) a processor configured to: (i) compare measured impedance values against baseline impedance values established at or shortly after implantation; (ii) calculate relative impedance change as a percentage of baseline; (iii) classify healing stage based on said relative impedance change;
(e) wherein said healing stage classification is derived from changes in tissue magnetic susceptibility at the fracture site as healing progresses from soft callus formation through mineralization to mature bone, said changes modulating magnetic coupling between said implanted permanent magnets and said external measurement coil.

5. (canceled)

6. The compact wearable PEMF therapy system of claim 1, wherein said electromagnetic coil has a diameter between 10 cm and 15 cm.

7. The compact wearable PEMF therapy system of claim 1, wherein said total mass is between 300 grams and 1000 grams.

8. The compact wearable PEMF therapy system of claim 1, wherein said rechargeable power source provides at least 8 hours of continuous therapy per charge cycle in implant-activation mode.

9. The compact wearable PEMF therapy system of claim 1, further comprising a wireless communication module configured to transmit therapy compliance data to a paired smartphone application.

10. The compact wearable PEMF therapy system of claim 1, further comprising a magnetometer configured to detect static magnetic fields from implanted permanent magnets, wherein said magnetometer output is used to confirm correct positioning of said external PEMF generator overlying implanted components.

11. The compact wearable PEMF therapy system of claim 1, wherein said wearable attachment mechanism comprises an elastic strap configured to wrap around a patient's limb.

12. The compact wearable PEMF therapy system of claim 1, wherein said housing has a disc-shaped form factor with maximum diameter between 10 cm and 18 cm and maximum thickness between 20 mm and 40 mm.

13. The compact wearable PEMF therapy system of claim 1, further comprising a USB-C charging interface.

14. The compact wearable PEMF therapy system of claim 1, wherein said pulse generation circuit generates frequencies between 5 Hz and 100 Hz.

15. The compact wearable PEMF therapy system of claim 9, wherein said smartphone application displays daily therapy compliance as a percentage of prescribed daily therapy duration.

16. The dual-mode PEMF therapy system of claim 2, wherein said implant-activation mode generates an external magnetic field of 3-10 mT and provides at least 24 hours of continuous operation per charge cycle.

17. The dual-mode PEMF therapy system of claim 2, wherein said standalone PEMF generation mode generates an external magnetic field of 8-20 mT.

18. The dual-mode PEMF therapy system of claim 2, wherein said mode selection mechanism comprises a physical button on the device housing.

19. The dual-mode PEMF therapy system of claim 2, wherein said mode selection mechanism is accessible via a paired smartphone application.

20. The dual-mode PEMF therapy system of claim 2, wherein said external PEMF generator has a disc-shaped housing with diameter between 10 cm and 18 cm and total mass between 300 grams and 1000 grams.

21. The combined electromagnetic therapy system of claim 4, wherein said implanted permanent magnets are neodymium magnets arranged in stacked configuration with alternating polarity.

22. The combined electromagnetic therapy system of claim 4, wherein said orthopaedic hardware is selected from the group consisting of: bone screws, intramedullary nails, bone pegs, and bone plates.

23-45. (canceled)
